(19) United States
(12) Patent Application Publication
(10) Pub. No.: US 2017/0259108 A1
(43) Pub. Date: Sep. 14, 2017

(54) WEIGHTED HANDHELD ELECTRONIC DEVICE

(71) Applicant: Jacob Feldstein, Princeton, NJ (US)

(72) Inventor: Jacob Feldstein, Princeton, NJ (US)

(21) Appl. No.: 15/066,820

(22) Filed: Mar. 10, 2016

(51) Int. Cl. A63B 21/072; A61B 5/00; A61B 5/024; A63B 24/00; A63B 21/06

(52) U.S. Cl. CPC A63B 21/0726; A63B 24/0062; A63B 21/0602; A63B 21/0603; A61B 5/02438; A61B 5/4866; A61B 5/6895; A61B 5/742; A63B 2230/06; A63B 2230/75

(57) ABSTRACT

The present invention is directed to a weighted hand held electronic device that is ergonomically shaped to fit one's hand. The device has the ability to control another device such as a television using built in remote control capabilities and the ability to control an auxiliary device such as a phone, music player, or other device that can be attached to this handheld device. The handheld device makes walking, running, or other forms of exercising easier and more productive while watching television because of the device's weight, convenience and functionality.

This application is a continuation-in-part of U.S. patent application Ser. No. 14/341,372, filed Jul. 25, 2014 and now pending, and incorporated herein by reference.

### BACKGROUND OF THE INVENTION

Physical exercise is very important and helpful to people. It improves health and the exerciser's mood and it burns fat, builds muscle, helps one's cardiac system, and it has many more advantages. There are many types of exercise that one can do such as walking, running, swimming, stair climbing, using an elliptical machine, lifting weights, resistance exercising, push-ups, pull ups, squats, yoga, and many others involving different types of motion and resistance. Leg exercises, such as walking, running, elliptical, stair climbing and others primarily help improve lower body strength and cardio functions. When one adds in upper body exercises such as lifting weights, it is more efficient in improving health because the cardiac work out increases and the user consequently exercises additional sections of the body. Lifting weights while doing lower body exercise is also time efficient as it exercises multiple sections of the body in the time it would otherwise take to exercise fewer sections. There are many types of equipment and weights used to increase resistance to the exerciser. One type of such a weight is a dumbbell.

Dumbbells are weights used for exercising. They may be used for many types of exercise, most of which involve lifting them or holding them. They may also be used when doing other exercises like walking or running. Dumbbells are typically shaped like a capital I and are available in different weights.

While exercising, exercisers often listen to music, audio books and other sources, or watch television or other visuals to keep entertained. This can be significant in allowing the individual to exercise longer than otherwise and therefore help the individual achieve greater results from their exercise.

Television remote control devices control a distribution source, such as a television or cable box, and are used to change the channel, volume, or other parameters on the television or other distribution source. Such a device does so by, for example sending a signal wirelessly to the television, which the television receives and does the command it is being told to do by the remote control.

Using a remote control while exercising can be problematic. Some problems which may be faced are small buttons, buttons that are hard to press while in motion, the need to look at the remote control instead of focusing on exercise or entertainment, a multitude of buttons not needed while exercising, where to place or hold the remote control, sweat getting onto or into the remote control which makes the remote control wet, slippery, unsanitary, and could cause malfunctions, and other problems are possible. Only some primary function buttons such as volume, channel, and power are needed while exercising. Typical remote controls are not sufficiently ergonomically designed, which makes remote controls hard to hold and the buttons hard to locate and press when in motion. Typical remote controls are not durable enough to survive a drop to a hard and/or moving exercise surface which is more likely to happen with a remote control that is not ergonomically designed, wet with sweat, and/or being held by an individual in motion. Moreover, typical remote controls are very light. Holding such a device in one's hand while exercising does not substantially add to one's exercise.

If an individual is exercising with dumbbells or other weights in one or both hands, and the person exercising wants to change some parameter on the television or other device, the person must put down the weights and pick up the television remote to affect the desired change. This may be either not convenient or not possible while an individual is exercising.

People running, walking and other exercising outdoors carry many types of devices like personal music players, mobile phones, geolocator devices, pedometers, other activity monitoring devices, heart monitors, and others. Some examples of these devices are Iphones, android phones, Ipods, GPS, MP3 players, Zoons, and others. These devices are used in order to make the physical activity more enjoyable, more effective, productive, and/or more convenient.

Currently people use a variety of means to hold these devices, such as armbands, waist belts, pockets, wrist bands, clips, and others. There is a potential problem faced with each of these methods. If the device is not hand held and/or not of sufficient weight, there will be no exercise benefit added. If the devices are not ergonomic, they can be hard to operate while exercising. For example, if the user reaches for a button while running, the user may find it difficult to position a finger on the button. If the device is in a pocket, clipped to the person's clothing, or within an armband, accessing the device may be difficult. These are just some examples of problems that one could encounter.

Using a complicated, inconvenient, non-ergonomic, electronic device while exercising can also be dangerous to the individual as it increases the risk of an accident while exercising. Using touch screen type devices can be especially complicated to use during physical activity as the user must often power or wake up the device, input a pass code, fingerprint scan, select one or more icons or other menu items, and then input the desired command. Consequently, there is an unmet need for a remote control device compatible with exercise activities and an unmet need for a weighted remote control to enhance the user's level of exercise during a workout.

Some attempts have been made to combine the attributes of the present invention, although unsuccessfully. For example, U.S. Pat. No. 6,042,508 describes an adjustable hand-held exercise weight with pulse detection and remote control capabilities. Among its disadvantages are that it can only fit one type of remote control that is already built in the device and it cannot hold other electronic devices. Also, the buttons are not in the most easily accessible location on the device and the shape does not ergonomically fit the shape of the hand.

Also, U.S. Pat. No. 5,910,070 describes a weighted remote control with the disadvantage that the user must reach his/her fingers to reach the buttons near the top of the device and it does not ergonomically fit the shape of the hand. It also is limited in other ways. Also, this device does not have a means of attaching an auxiliary device.

### BRIEF DESCRIPTION OF THE PRESENT INVENTION The present invention is directed to a multi-purpose device, usable during exercise, which concurrently solves the numerous issues identified above.

The present invention is primarily directed to a hand-held device, such as but not limited to a dumbbell, which one holds during lower body exercise. Among its beneficial features, which may be included in their entirety or as a subset, include:
Weighted
Ergonomic shape
Exterior material used to limit the likelihood of slippage, especially consequential to exercise
Finger grooves for holding the device, including grooves on both sides
Buttons in finger grooves, where the buttons can be configured for controlling different devices or functions. Buttons can be used to control internal or external elements. Buttons may be engaged without the user relocating a finger.
The buttons may be programmable through an interface
A microphone for receiving content
Finger grooves arranged for use in either a left or right hand
Internal battery for powering device or charging an element stored in the device. Battery is rechargeable.
Port for physically attaching another device, such as a television remote control or a mobile phone.
Wireless port for communicating with remote devices
Speaker for audibly relaying content
Removable compartment for storing drinking or hydrating fluid. Spout or other drinking port controllable by button.
Drinking compartment configured to limit movement of internal fluid.
Internal bladder for drinking compartment
Internal container for electronic device
Internal container for adding weight
Internal compartment for delivering solids for digestion

### DESCRIPTION OF THE FIGURES

FIG. 1 depicts the weighted device of the present invention in the form of a dumbbell shape.

FIG. 2 depicts a side perspective schematic view of an embodiment of the weighted device of the present invention, showing an embodiment with three separate storage compartments.

FIG. 3 depicts a side perspective schematic view of an embodiment of the weighted device of the present invention, showing an embodiment with three separate storage compartments and showing the opposite side from that of FIG. 2.

### DETAILED DESCRIPTION OF THE INVENTION

The present invention is directed to meeting the unmet need of making hand-held devices used for improving conditioning while exercising being used to encompass features often needed by exercisers which preclude continuous exercise use. For example, a user may wish to take a drink or change a channel on a television. The present invention is further usable for making remote control devices compatible with and beneficial for physical activity such as exercise.

The weighted, handheld electronic device (“weighted control device”) of the present invention can be in the shape of a dumbbell, or be cylindrical, grooved, smooth, or any other shape such that it can be easily hand held while exercising. The exterior can be made out of rubber, plastic, vinyl, metal and/or other materials. In the preferred embodiment, the device is shaped like a dumbbell with finger grooves and its exterior surface is formed of a rubberized and spongy material so as to provide the benefits of ease of holding, especially as the hand becomes moist from exercise, and absorbency. The device includes a processor and memory so as to allow for programming of control elements included in the device.

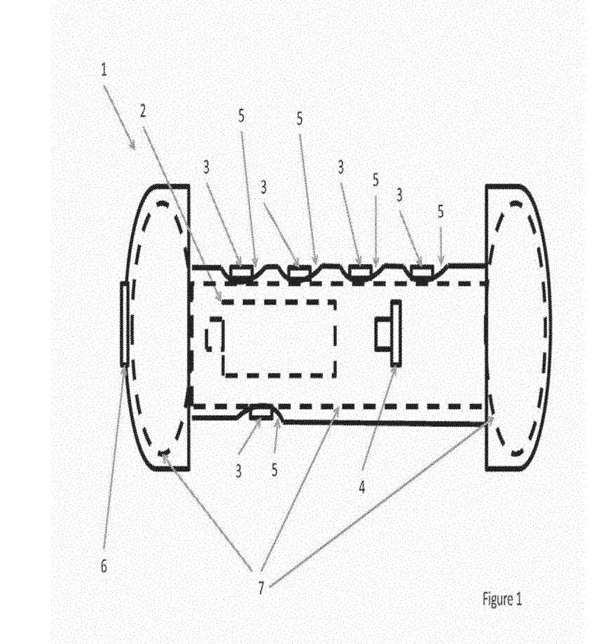

FIG. 1 is a side view of the weighted handheld electronic device 1. The weighted handheld electronic device 1 preferably has five ergonomically shaped ridges 5 that each has one button 3 inside the finger areas. Preferably, the ridges are positioned for a person's fingers—that is, four slots on one side and one on the other for a thumb. In at least some embodiments, the device may have two thumb slots so as to allow use in either one's left or right hand. The buttons 3 control a secondary electronic device. A battery 2 that can also be the power source to the auxiliary device that is attached to the attachment site 4 and the buttons 3. Additional weight may be added consequential to material like sand or water or other solids or liquids that will be poured through the compartment-filling cap 6 into a compartment 7.

Battery 2 can also be used to power the device itself or an element of the device and is potentially rechargeable. For example, a refrigeration coil or a heating coil can be included in the device, powered by battery 2, so as to, for example, control defrosting speed of a frozen drink embedded in the device.

The device can have an adapter to receive/hold an auxiliary device such as a television remote control, mp3 player, phone, or others; and/or the device can have its own built-in buttons, switches, knobs or other control features which can be used to send a signal to another device. In the present invention, these built in buttons, switches, knobs, or other control features can be positioned in ridges made for fingers that would be ergonomically shaped to fit one's hand and fingers. If these control features are built in to the weighted control device, there would not need to be as many buttons as on a standard remote control, which would make this ergonomic and weighted remote control device simpler and easier to use.

The signaling capability on the weighted remote control device can control functions on either a television (or cable box, among others) remote device or on an auxiliary device that is adapted to the weighted remote control device. There may be a compartment, slot, adapter or other element where one may connect or mate with a phone or other device to the weighted remote control and still use the device while attached. In addition or in the alternative, the signaling capability can control an electronic device housed within the weighted handheld device and/or connected to the weighted handheld device. That is, any or all of the buttons may control devices internally or externally, and different buttons can control different devices or even different functions of different devices.

The signaling capability may be enhanced by being two way. For example, data can be delivered to the device of the present invention with indication of receipt, such as by audible indicator. The audible (or other) indicator may be included in the device itself or in an auxiliary element mated with the device.

The weighted remote control can be any weight above that of a conventional remote control device. The device would preferably be 1 to 10 pounds in weight, more preferably 2 to 5 pounds, as is common for individuals to hold during exercise. The specific weight depends on the ability and objectives of the individual user. The device could get most of its weight from an internal solid or liquid. For example, the device could include an internal compartment which could be filled with either sand, water, gel, or a material of any type that would make the device heavier. The device could get its weight from being filled up with those materials, in which case, it could also be emptied and be less heavy, which would make it easier for travel, or it could have permanent internal weight. The device would be good for travel, good for customizing to a desired weight if being filled up, and if it is filled with water or another liquid, it could be a good source of water or other liquid to drink for hydration or to cool one's self off with. The weight may also be consequential to an internal large battery that can be used for additional power to the remote and/or auxiliary devices. The device could have integrated electronics or controls. The device can have a compartment, to which an auxiliary electronic device may be attached. The device could have a mating connector for portable media players, GPS devices, phones, transmitters of any type, or anything else. The buttons on the remote are large and easily engageable. The buttons could be, but may not be limited to; the volume up and down buttons, the channel up and down buttons, and the power button.

A weighted remote control device according to the present invention could have built in audio speakers to add weight to the remote control as well as provide audio to the user that is transmitted from an adapted electronic device such as an mp3 device, phone, GPS device, or others.

The accompanying figures illustrate a preferred embodiment of a weighted handheld electronic device and respective constituent parts, however, other types and styles are possible, and the drawing is not intended to be limiting in that regard. Thus, although the description above and the accompanying drawings contain much specificity, the details provided should not be construed as limiting the scope of the embodiment but merely as providing illustrations of some of the presently preferred embodiment. The drawings and the description are not to be taken as restrictive on the scope of the embodiment and are understood as broad and general teachings in accordance with the present invention. While the present embodiment of the invention has been described using specific terms, such description is for present illustrative purposes only, and it is to be understood that modifications and variations to such embodiment, including but not limited to the substitutions of equivalent features, materials, or parts, and the reversal of various features thereof, may be practiced by those of ordinary skill in the art without departing from the spirit and scope of the invention.

In the preferred embodiment, the weighted control device of the present invention is ergonomically shaped to compliment the human hand and includes a gripper portion. The exterior is formed of plastic or rubber. The device includes ergonomically placed buttons. The weight is or may be adjustable based on the actual composition of the device. The weight largely is consequential to an internal rechargeable battery that can power the handheld device as well as any connected electronic device. The weight may also be consequential to fixed materials within the device for the purpose of adding weight to the device. The weight may also be consequential to addition of other materials such as solids or liquids that can be added to the device on a temporary basis.

The weighted control device of the present invention includes a connection for another electronic device that can attach to it either directly or via an adapter. A compartment or other means of connection for an auxiliary electronic device may be included or attached to the handheld device. The auxiliary electronic device may be able to operate off of the battery in the weighted control device, and the auxiliary electronic device may be able to charge its own internal battery from the battery in the handheld electronic device. One or more buttons on the handheld electronic device are placed at or around the fingertips of when the device is gripped. Additional weight can be added from water or another liquid or solid material that can temporarily or permanently be housed in an internal compartment. That compartment can be opened up like a canteen and include a spout to allow the user to drink liquid for hydration while exercising. The weighted control device is waterproof or water resistant so that the water will not affect the device or an attached auxiliary device. The buttons on the device may control volume up and down, channel up and down, and power on and off. The buttons can operate as a remote control for another device such as a video or audio device or provide other controls. The buttons can also operate an auxiliary device that is attached or connected to the handheld device. For example, the buttons could control the volume up and down, and next and previous song of a music-playing device when attached to the handheld device. The buttons can also control the volume of a phone that is attached to the handheld device.

The material for any portions of the device that would be in contact with a liquid useable for drinking would be suitable for this purpose. Specifically the liquid contact areas could be stainless steel, glass, or a safer plastic typically used for potable water bottles and other devices such as Polyethylene Terephthalate PET or PETE, high density polyethylene, HDPE, low density polyethylene LDPE, or polypropylene PP. In addition any of these materials could be treated to be anti-bacterial to make the device even safer for the user to drink from.

The compartment for containing a liquid may be opened or accessed through a cap, lid, flip valve, valve which opens and closes with the push of one of the finger buttons on the handheld device, through a straw or hose which may have a check valve and/or bite valve to allow the user to receive the liquid without fluids or any other materials flowing back into the liquid compartment. There may be an additional second access point, such as being a cap, lid, or other device to allow easier access to the internal compartment of the handheld device and to potentially make it entirely removable. This second access point could have a wider opening than the first access point, so as to improve the ability to pour fluids or other flowables into the container. This second access point provides the benefit of better cleanability including the potential to make the inner liquid chamber removable and dishwasher safe. The inner compartment may also be a shape and configuration that is adaptable for an insertable bladder containing a liquid, gel, or frozen liquid for consumption by the user during exercise.

In addition, another inner compartment may be configured to be useable for dispensing edible products such as energy pills, jelly beans, candy, and other solid edible products consumed by users while exercising.

One or more of the inner compartments may be insulated using known forms of insulation to keep any contained liquids, gels, frozen liquids, or solids at a desired temperature; for example cold materials for drinking and refreshment, or warm materials to keep the hands of the user warm during winter exercise. For example, a surrounding area for ice or surrounding insulation may be included.

The handheld device may be constructed to generate a vibration, light, sound, or other signal when a mated auxiliary device such as a mobile phone or others described in this application receives an incoming signal such as an email, text, phone call, or other notification. The handheld device of the present invention may be equipped with one or more audio speakers as well as Bluetooth or alternative wireless communication technology in order to connect the handheld device wirelessly to a television, computer, or other paired device to play sound from the paired device on the speaker or speakers of the handheld device, thereby enabling the user to hear the audio more clearly and directly.

The handheld device may contain the necessary equipment and software to provide heart rate monitoring and/or identify calories burned data to the user from the handheld device and/or an auxiliary device connected to the handheld exercise device.

The weighting of the handheld device may be configured in a way as to be elastic or movable for added resistance and engagement of muscles during motion in multiple directions for added upper body conditioning.

In at least one embodiment of the present invention, the device may have a plurality of compartments with different compartments suitable for different purposes, such as for storing beverages, solids for ingesting, weights, a remote control device, and other items. Each compartment may be configured for a different purpose, and each compartment may be shaped differently or sized differently from other compartments. For example, a compartment for fluids may be shaped so as to preclude (or encourage) fluid movement within the device.

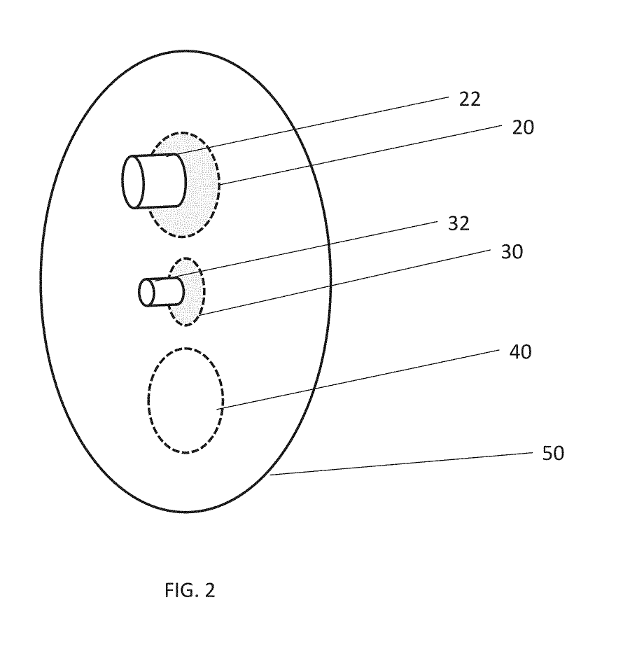

FIG. 2 depicts a side perspective view of one end of one embodiment of the present invention, particularly showing an embodiment with three distinct compartments. Side of weighted remote 50 includes three internal compartments. Liquids compartment 20, which could be configured for holding fluid, includes liquids spout 22. A user could, for example, fill liquid compartment 20 with water or an energy drink and drink from liquids spout 22. Liquids spout 22 could be opened by, for example, one of the finger controllers of the present invention, whereby a user could push the controller once to open and again to close, or by some other known method.

Liquids compartment 20 can be structured to hold liquids at various temperatures and could be insulated.

Solids compartment 30 could be used for solids, such as energy jelly beans, and could be configured to deliver one bean (or equivalent) at a time. Solids spout 32 could be used for delivery and like solids spout 32, could be controllable by the finger slots.

Although shown as being elevated from the surface, each spout could be structured to be elevated by having a rotatable section, with the opening and closing of the spout separately controllable.

Weight compartment 40 could be used for storing an element to add weight to the device, where the element may be a hard weight or may be flowable such as sand.

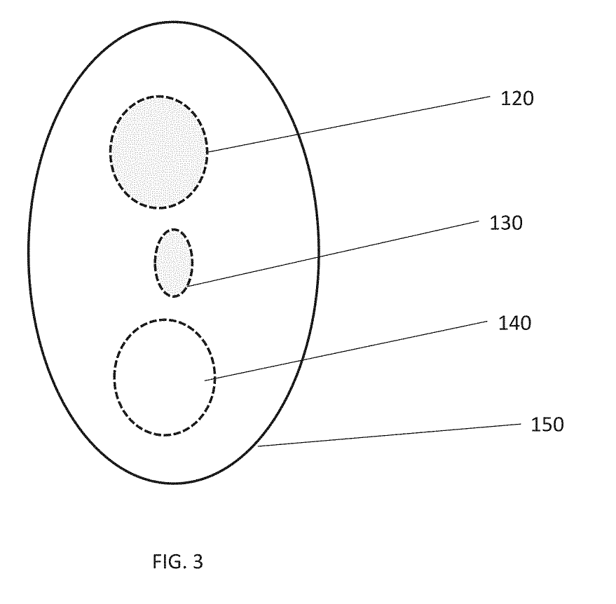

FIG. 3 shows the opposite side 150 of the same embodiment as shown in FIG. 2. Fluids compartment 20 has an opposite side wide-mouth opening 120. Fluid can be poured into fluids compartment 20 by the wide-mouth fluids opening 120 shown in FIG. 3. Similarly, solids compartment 30 has a wide-mouth solids opening 130 and weight compartment 40 has a wide-mouth weight opening 140. As can be envisioned with FIG. 3, each of the three compartments may be removable and can be inserted in place directly with some form of lock down (not shown) or by being screwed into place. Each of the three may be removed for cleaning.

Although not shown, other compartments can be included in the device as well, with different compartments serving different purposes. For example, a slot for a plug-in device, such as a remote controller for a television or a slot for a mobile phone can be included, with the slot including a means for connecting the inserted device with the weighted control device.

1. A grippable multi-functional exercise apparatus for improving upper body conditioning while lower body exercising and which controls at least one electronic appliance comprising:
a gripper element comprising a plurality of finger grooves with at least one of said grooves containing a button for engaging a signal, said finger grooves distributed so as to facilitate holding while exercising;
at least one storage compartment with an opening, said opening controllable with a button in a finger groove;
an internal battery configurable to power an electronic device mated with said apparatus;
a port for mating an electronic device; and
a signaling element for communicating with an electronic appliance with signaling triggered by a user engaging said at least one button.

2. The apparatus of claim 1, wherein said apparatus is dumbbell shaped.

3. The apparatus of claim 1, wherein said at least one storage compartment includes a sealable cavity usable for storing liquid or flowing solid.

4. The apparatus of claim 3, wherein said compartment interior is formed with non-toxic material selected from the group comprising: stainless steel, glass, polyethylene tetraphthalate (PET or PETE), high density polyethylene (HDPE), low density polyethylene (LDPE), and polypropylene (PP).

5. The apparatus of claim 3, wherein said compartment is accessible by a person using a controllable valve which opens and closes with the push of one of said buttons.

6. The apparatus of claim 5, further including a check or bite valve to allow the user to receive the liquid without fluids or flowing solids.

7. The apparatus of claim 5, wherein said sealable cavity may be accessed through two or more openings.

8. The apparatus of claim 3, wherein said sealable cavity is removable and dishwasher safe.

9. The apparatus of claim 3, wherein said sealable cavity is adaptable for an insertable bladder internally including liquid, gel, or frozen liquid.

10. The apparatus of claim 9, wherein said sealable cavity is insulated to keep said contents at a desired temperature.

11. The apparatus of claim 3, wherein said apparatus further includes a spout in communication with said cavity.

12. The apparatus of claim 1, wherein said battery is rechargeable.

13. The apparatus of claim 1, wherein said electronic device is one of the set of a phone, a music storage, a player device, and a GPS device and the audio output of said device is delivered through said apparatus.

14. The apparatus of claim 1, wherein said apparatus includes a plurality of buttons in said finger grooves for signal control of a remote electronic appliance including, one button to control remote volume, one button to control channel selection, and one button to control power of a remote electronic device.

15. The apparatus of claim 1, wherein said apparatus is configurable to notify the user of notifications from said additional electronic device by generating a signal which selected from the group comprising a vibration, a light, and sound.

16. The apparatus of claim 1, wherein said port is a wireless port.

17. The apparatus of claim 16, further including an audio speaker.

18. The apparatus of claim 1, further comprising means to measure and deliver a user's heart rate or calories burned for display.
